(19) United States
(12) Patent Application Publication
(10) Pub. No.: US 2022/0096535 A1
(43) Pub. Date: Mar. 31, 2022

(54) COMBINATION DRUG THERAPIES OF PDE-5 INHIBITORS AND INHALED NITRIC OXIDE

(71) Applicant: BELLEROPHON THERAPEUTICS, Warren, NJ (US)

(72) Inventors: Parag SHAH, Morristown, NJ (US); Peter Paul FERNANDES, Randolph, NJ (US); Bobae KIM, Morristown, NJ (US)

(73) Assignee: BELLEROPHON THERAPEUTICS, Warren, NJ (US)

(21) Appl. No.: 17/421,803

(22) PCT Filed: Jan. 14, 2020
(86) PCT No.: PCT/US20/13446

§ 371 Date: Jul. 9, 2021

Related U.S. Application Data
(60) Provisional application No. 62/792,329, filed on Jan. 14, 2019.

(51) Int. Cl. A61K 33/00; A61M 16/00; A61K 31/519; A61K 31/4985; A61K 31/53; A61K 31/506; A61K 31/522; A61P 9/12

(52) U.S. Cl. CPC A61K 33/00; A61M 16/022; A61K 31/519; A61K 31/4985; A61M 2202/0275; A61K 31/506; A61K 31/522; A61P 9/12; A61M 2230/42; A61K 31/53

(57) ABSTRACT

Described are methods for treatment of hypertension in combination with pulsed, inhaled nitric oxide.

### CROSS REFERENCE TO RELATED APPLICATIONS

This application claims the benefit of U.S. Provisional Patent Application No. 62/792,329 filed Jan. 14, 2019 entitled “Combination Drug Therapies of PDE-5 Inhibitors and Inhaled Nitric Oxide”, which is incorporated by reference herein in its entirety.

### FIELD OF THE INVENTION

The present application relates generally to combination therapies of PDE-5 inhibitors and inhaled nitric oxide (iNO).

### BACKGROUND OF THE INVENTION

Nitric oxide (NO) is a gas that, when inhaled, acts to dilate blood vessels in the lungs, improving oxygenation of the blood and reducing pulmonary hypertension. NO acts on the cGMP pathway causing dilation of the blood vessels in the lungs. Because of this, nitric oxide is provided as a therapeutic gas in the inspiratory breathing phase for patients having difficulty breathing due to a disease state, for example, pulmonary arterial hypertension (PAH), chronic obstructive pulmonary disorder (COPD), cystic fibrosis (CF), idiopathic pulmonary fibrosis (IPF), emphysema, or other lung disease.

While NO may be therapeutically effective when administered under the appropriate conditions, it can also become toxic if not administered correctly. NO reacts with oxygen to form nitrogen dioxide (NO₂), and NO₂ can be formed when oxygen or air is present in the NO delivery conduit. NO₂ is a toxic gas which may cause numerous side effects, and the Occupational Safety & Health Administration (OSHA) provides that the permissible exposure limit for general industry is only 5 ppm. Thus, it is desirable to limit exposure to NO₂ during NO therapy.

Effective dosing of NO is based on a number of different variables, including quantity of drug and the timing of delivery. Several patents have been granted relating to NO delivery, including U.S. Pat. Nos. 7,523,752; 8,757,148; 8,770,199; and 8,803,717, and a Design Patent D701,963 for a design of an NO delivery device, all of which are herein incorporated by reference. Additionally, there are pending applications relating to delivery of NO, including US2013/0239963 and US2016/0106949, both of which are herein incorporated by reference. Even in view of these patents and pending publications, there is still a need for methods and apparatuses that deliver NO in a precise, controlled manner, so as to maximize the benefit of a therapeutic dose and minimize the potentially harmful side effects.

Pharmaceutical compositions used to treat hypertension and other vasodilators, such as riociguat or inhibitors of cGMP-specific phosphodiesterase type 5 (PDE-5) like sildenafil and tadalafil, are used in the treatment of pulmonary arterial hypertension (PAH) due to their vasodilative effects. Many marketed vasodilators (including riociguat and PDE-5 inhibitors) are currently labeled with a warning against use of nitrates while taking such drug due to the risk of hypotension. As NO is a source of nitrate, NO would typically not be used as a combination therapy with PDE-5 inhibitors. The present invention demonstrates that use of iNO has no additive hemodynamic effect when co-administered with hypertension drug treatments, and iNO is therefore safe to co-administer with such vasodilators.

### SUMMARY OF THE INVENTION

In an embodiment, a method for preventing an additive hemodynamic effect when treating a patient with a therapeutically effective amount of a pharmaceutical composition for treatment of hypertension, the method comprising co-administering with said pharmaceutical composition a therapeutically effective amount of inhaled nitric oxide is described herein.

In one embodiment, the nitric oxide is delivered in a pulsatile manner by detecting a breath pattern in said patient including a total inspiratory time, correlating the breath pattern with an algorithm to calculate the timing of administration of the dose of nitric oxide; and delivering the nitric oxide to the patient in a pulsatile manner over a portion of the total inspiratory time. In one embodiment, the dose of nitric oxide occurs within the first half of the total inspiratory time. In another embodiment, the nitric oxide is delivered in a series of pulses over a period of time.

In one embodiment, the pharmaceutical composition is a PDE-5 inhibitor. In another embodiment, the PDE-5 inhibitor is sildenafil. In another embodiment, the PDE-5 inhibitor is tadalafil. In another embodiment, the PDE-5 inhibitor is vardenafil. In another embodiment, the PDE-5 inhibitor is a non-specific PDE-5 inhibitor such as dipyridamole or theophylline. In yet another embodiment, the pharmaceutical composition is a stimulator of soluble guanylate cyclase. In another embodiment, the pharmaceutical composition is riociguat.

In an embodiment of the present invention, a method for preventing a systemic, additive decrease in blood pressure in a patient being treated for hypertension while maintaining an improvement in the overall treatment of said hypertension, the method comprising co-administering a PDE-5 inhibitor and inhaled nitric oxide is described herein. In one embodiment, the nitric oxide is delivered by detecting a breath pattern in the patient, said breath pattern having a total inspiratory time and a total expiratory time; correlating the breath pattern with an algorithm to calculate the timing of administration of a dose of nitric oxide; and delivering said dose of nitric oxide to said patient in a pulsatile manner over a portion of the total inspiratory time for a period of time required for a therapeutically effective amount of nitric oxide to be delivered to said patient. In one embodiment, the PDE-5 inhibitor is sildenafil. In another embodiment, the PDE-5 inhibitor is tadalafil.

### BRIEF DESCRIPTION OF THE DRAWINGS

The foregoing summary, as well as the following detailed description of the invention, will be better understood when read in conjunction with the appended drawings.

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIGS. 1-5 are graphs demonstrating systolic and diastolic blood pressure (in mmHg) for five different patients receiving sildenafil and iNO treatments at various time points at dosing, pre-dosing with sildenafil, and post-dosing with sildenafil. The top plot represents systolic measurements, the bottom plot represents diastolic measurements, the diastolic cut off is 60 mmHg, the systolic cut off is 90 mmHg, and the lines within each plot represent the linear systolic and diastolic pressures across the entire dosing period.

FIG. 6 is a graph demonstrating the average iNO metabolites for all patients (mean plus standard deviation bars) over time. Also shown is administration of seven doses of sildenafil (spikes at bottom of graph), and co-administration of iNO with doses 4-7 of sildenafil (dotted line).

FIG. 7 illustrates the protocol design for Example 1.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

Before describing several exemplary embodiments of the invention, it is to be understood that the invention is not limited to the details of construction or process steps set forth in the following description. The invention is capable of other embodiments and of being practiced or being carried out in various ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

### DEFINITIONS

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

When ranges are used herein to describe an aspect of the present invention, for example, dosing ranges, amounts of a component of a formulation, etc., all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

With respect to the present invention, in certain embodiments, a dose of a gas (e.g., NO) is administered in a pulse to a patient during an inspiration by the patient. It has been surprisingly discovered that nitric oxide delivery can be precisely and accurately delivered within the first two-thirds of total breath inspiration time and the patient obtains benefits from such delivery. Such delivery minimizes loss of drug product and risk of detrimental side effects increases the efficacy of a pulse dose which in turn results in a lower overall amount of NO that needs to be administered to the patient in order to be effective. Such delivery is useful for the treatment of various diseases, such as but not limited to idiopathic pulmonary fibrosis (IPF), pulmonary arterial hypertension (PAH), including Groups I-V pulmonary hypertension (PH), chronic obstructive pulmonary disorder (COPD), cystic fibrosis (CF), and emphysema, and is also useful as an antimicrobial, for example, in treating pneumonia.

Such precision has further advantages in that only portions of the poorly ventilated lung area is exposed to NO. Hypoxia and issues with hemoglobin may also be reduced with such pulsed delivery, while NO₂ exposure is also more limited.

### Breath Patterns, Detection and Triggers

Breath patterns vary based on the individual, time of day, level of activity, and other variables; thus it is difficult to predetermine a breath pattern of an individual. A delivery system that delivers therapeutics to a patient based on breath pattern, then, should be able to handle a range of potential breath patterns in order to be effective.

In certain embodiments, the patient or individual can be any age, however, in more certain embodiments the patient is sixteen years of age or older.

In an embodiment of the invention, the breath pattern includes a measurement of total inspiratory time, which as used herein is determined for a single breath. However, depending on context “total inspiratory time” can also refer to a summation of all inspiratory times for all detected breaths during a therapy. Total inspiratory time may be observed or calculated. In another embodiment, total inspiratory time is a validated time based on simulated breath patterns.

In an embodiment of the invention, breath detection includes at least one and in some embodiments at least two separate triggers functioning together, namely a breath level trigger and/or a breath slope trigger.

In an embodiment of the invention, a breath level trigger algorithm is used for breath detection. The breath level trigger detects a breath when a threshold level of pressure (e.g., a threshold negative pressure) is reached upon inspiration.

In an embodiment of the invention, a breath slope trigger detects breath when the slope of a pressure waveform indicates inspiration. The breath slope trigger is, in certain instances, more accurate than a threshold trigger, particularly when used for detecting short, shallow breaths.

In an embodiment of the invention, a combination of these two triggers provides overall a more accurate breath detection system, particularly when multiple therapeutic gases are being administered to a patient simultaneously.

In an embodiment of the invention, the breath sensitivity control for detection of either breath level and/or breath slope is fixed. In an embodiment of the invention, the breath sensitivity control for detection of either breath level or breath slope is adjustable or programmable. In an embodiment of the invention, the breath sensitivity control for either breath level and/or breath slope is adjustable from a range of least sensitive to most sensitive, whereby the most sensitive setting is more sensitive at detecting breaths than the least sensitive setting.

In certain embodiments where at least two triggers are used, the sensitivity of each trigger is set at different relative levels. In one embodiment where at least two triggers are used, one trigger is set a maximum sensitivity and another trigger is set at less than maximum sensitivity. In one embodiment where at least two triggers are used and where one trigger is a breath level trigger, the breath level trigger is set at maximum sensitivity.

Oftentimes, not every inhalation/inspiration of a patient is detected to then be classified as an inhalation/inspiration event for the administration of a pulse of gas (e.g., NO). Errors in detection can occur, particularly when multiple gases are being administered to a patient simultaneously, e.g., NO and oxygen combination therapies.

Embodiments of the present invention, and in particular an embodiment which incorporates a breath slope trigger alone or in combination with another trigger, can maximize the correct detection of inspiration events to thereby maximize the effectiveness and efficiency of a therapy while also minimizing waste due to misidentification or errors in timing.

In certain embodiments, greater than 50% of the total number of inspirations of a patient over a timeframe for gas delivery to the patient are detected. In certain embodiments, greater than 75% of the total number of inspirations of a patient are detected. In certain embodiments, greater than 90% of the total number of inspirations of a patient are detected. In certain embodiments, greater than 95% of the total number of inspirations of a patient are detected. In certain embodiments, greater than 98% of the total number of inspirations of a patient are detected. In certain embodiments, greater than 99% of the total number of inspirations of a patient are detected. In certain embodiments, 75% to 100% of the total number of inspirations of a patient are detected.

### Dosages and Dosing Regimens of NO

In an embodiment of the invention, nitric oxide delivered to a patient is formulated at concentrations of about 3 to about 18mg NO per liter, about 6 to about 10 mg per liter, about 3 mg NO per liter, about 6 mg NO per liter, or about 18 mg NO per liter. The NO may be administered alone or in combination with an alternative gas therapy. In certain embodiments, oxygen (e.g., concentrated oxygen) can be administered to a patient in combination with NO.

In an embodiment of the present invention, a volume of nitric oxide is administered (e.g., in a single pulse) in an amount of from about 0.350 mL to about 7.5 mL per breath. In some embodiments, the volume of nitric oxide in each pulse dose may be identical during the course of a single session. In some embodiments, the volume of nitric oxide in some pulse doses may be different during a single timeframe for gas delivery to a patient. In some embodiments, the volume of nitric oxide in each pulse dose may be adjusted during the course of a single timeframe for gas delivery to a patient as breath patterns are monitored. In an embodiment of the invention, the quantity of nitric oxide (in ng) delivered to a patient for purposes of treating or alleviating symptoms of a pulmonary disease on a per pulse basis (the “pulse dose”) is calculated as follows and rounded to the nearest nanogram value:

Dose ug/kg-IBW/hr×Ideal body weight in kg (kg-IBW)×((1 hr/60 min)×(1 min/respiratory rate (bpm))×(1,000 ng/ug).

As an example, Patient A at a dose of 100 ug/kg IBW/hr has an ideal body weight of 75kg, has a respiratory rate of 20 breaths per minute (or 1200 breaths per hour):

100 ug/kg-IBW/hr×75 kg×(1 hr/1200 breaths)×(1,000 ng/ug)=6250 ng per pulse

In certain embodiments, the 60/respiratory rate (ms) variable may also be referred to as the Dose Event Time. In another embodiment of the invention, a Dose Event Time is 1 second, 2 seconds, 3 seconds, 4 seconds, 5 seconds, 6 seconds, 7 seconds, 8 seconds, 9 seconds, or 10 seconds.

In an embodiment of the invention, a single pulse dose provides a therapeutic effect (e.g., a therapeutically effective amount of NO) to the patient. In another embodiment of the invention, an aggregate of two or more pulse doses provides a therapeutic effect (e.g., a therapeutically effective amount of NO) to the patient.

In an embodiment of the invention, at least about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 625, about 650, about 675, about 700, about 750, about 800, about 850, about 900, about 950, or about 1000 pulses of nitric oxide is administered to a patient every hour.

In an embodiment of the invention, a nitric oxide therapy session occurs over a timeframe. In one embodiment, the timeframe is at least about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours per day.

In an embodiment of the invention, a nitric oxide treatment is administered for a timeframe of a minimum course of treatment. In an embodiment of the invention, the minimum course of treatment is about 10 minutes, about 15 minutes, about 20 minutes, about 30 minutes, about 40 minutes, about 50 minutes, about 60 minutes, about 70 minutes, about 80 minutes, or about 90 minutes. In an embodiment of the invention, the minimum course of treatment is about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours. In an embodiment of the invention, the minimum course of treatment is about 1, about 2, about 3, about 4, about 5, about 6, or about 7 days, or about 1, about 2, about 3, about 4, about 5, about 6, about 7, or about 8 weeks, or about 1, about 2, about 3, about 4, about 5, about 6, about 7, about 8, about 9, about 10, about 11, about 12, about 18, or about 24 months.

In an embodiment of the invention, a nitric oxide treatment session is administered one or more times per day. In an embodiment of the invention, nitric oxide treatment session may be once, twice, three times, four times, five times, six times, or more than six times per day. In an embodiment of the invention, the treatment session may be administered once a month, once every two weeks, once a week, once every other day, daily, or multiple times in one day.

### Timing of a Pulse of NO

In an embodiment of the invention, the breath pattern is correlated with an algorithm to calculate the timing of administration of a dose of nitric oxide.

The precision of detection of an inhalation/inspiration event also permits the timing of a pulse of gas (e.g., NO) to maximize its efficacy by administering gas at a specified time frame of the total inspiration time of a single detected breath.

In an embodiment of the invention, at least fifty percent (50%) of the pulse dose of a gas is delivered over the first third of the total inspiratory time of each breath. In an embodiment of the invention, at least sixty percent (60%) of the pulse dose of a gas is delivered over the first third of the total inspiratory time. In an embodiment of the invention, at least seventy-five percent (75%) of the pulse dose of a gas is delivered over the first third of the total inspiratory time for each breath. In an embodiment of the invention, at least eighty-five (85%) percent of the pulse dose of a gas is delivered over the first third of the total inspiratory time for each breath. In an embodiment of the invention, at least ninety percent (90%) of the pulse dose of a gas is delivered over the first third of the total inspiratory time. In an embodiment of the invention, at least ninety-two percent (92%) of the pulse dose of a gas is delivered over the first third of the total inspiratory time. In an embodiment of the invention, at least ninety-five percent (95%) of the pulse dose of a gas is delivered over the first third of the total inspiratory time. In an embodiment of the invention, at least ninety-nine (99%) of the pulse dose of a gas is delivered over the first third of the total inspiratory time. In an embodiment of the invention, 90% to 100% of the pulse dose of a gas is delivered over the first third of the total inspiratory time.

In an embodiment of the invention, at least seventy percent (70%) of the pulse dose is delivered to the patient over the first half of the total inspiratory time. In yet another embodiment, at least seventy-five percent (75%) of the pulse dose is delivered to the patient over the first half of the total inspiratory time. In an embodiment of the invention, at least eighty percent (80%) of the pulse dose is delivered to the patient over the first half of the total inspiratory time. In an embodiment of the invention, at least 90 percent (90%) of the pulse dose is delivered to the patient over the first half of the total inspiratory time. In an embodiment of the invention, at least ninety-five percent (95%) of the pulse dose is delivered to the patient over the first half of the total inspiratory time. In an embodiment of the invention, 95% to 100% of the pulse dose of a gas is delivered over the first half of the total inspiratory time

In an embodiment of the invention, at least ninety percent (90%) of the pulse dose is delivered over the first two-thirds of the total inspiratory time. In an embodiment of the invention, at least ninety-five percent (95%) of the pulse dose is delivered over the first two-thirds of the total inspiratory time. In an embodiment of the invention, 95% to 100% of the pulse dose is delivered over the first two-thirds of the total inspiratory time.

When aggregated, administration of a number of pulse doses over a therapy session/timeframe can also meet the above ranges. For example, when aggregated greater than 95% of all the pulse doses administered during a therapy session were administered over the first two thirds of all of the inspiratory times of all of the detected breaths. In higher precision embodiments, when aggregated greater than 95% of all the pulse doses administered during a therapy session were administered over the first third of all of the inspiratory times of all of the detected breaths.

Given the high degree of precision of the detection methodologies of the present invention, a pulse dose can be administered during any specified time window of an inspiration. For example, a pulse dose can be administered targeting the first third, middle third or last third of a patient's inspiration. Alternatively, the first half or second half of an inspiration can be targeted for pulse dose administration. Further, the targets for administration may vary. In one embodiment, the first third of an inspiration time can be targeted for one or a series of inspirations, where the second third or second half may be targeted for one or a series of subsequent inspirations during the same or different therapy session. Alternatively, after the first quarter of an inspiration time has elapsed the pulse dose begins and continues for the middle half (next two quarters) and can be targeted such that the pulse dose ends at the beginning of the last quarter of inspiration time. In some embodiments, the pulse may be delayed by 50, 100, or 200 milliseconds (ms) or a range from about 50 to about 200 milliseconds.

The utilization of a pulsed dose during inhalation reduces the exposure of poorly ventilated areas of the lung and alveoli from exposure to a pulsed dose gas, e.g., NO. In one embodiment, less than 5% of poorly ventilated (a) areas of the lung or (b) alveoli are exposed to NO. In one embodiment, less than 10% of poorly ventilated (a) areas of the lung or (b) alveoli are exposed to NO. In one embodiment, less than 15% of poorly ventilated (a) areas of the lung or (b) alveoli are exposed to NO. In one embodiment, less than 20% of poorly ventilated (a) areas of the lung or (b) alveoli are exposed to NO. In one embodiment, less than 25% of poorly ventilated (a) areas of the lung or (b) alveoli are exposed to NO. In one embodiment, less than 30% of poorly ventilated (a) areas of the lung or (b) alveoli are exposed to NO. In one embodiment, less than 50% of poorly ventilated (a) areas of the lung or (b) alveoli are exposed to NO. In one embodiment, less than 60% of poorly ventilated (a) areas of the lung or (b) alveoli are exposed to NO. In one embodiment, less than 70% of poorly ventilated (a) areas of the lung or (b) alveoli are exposed to NO. In one embodiment, less than 80% of poorly ventilated (a) areas of the lung or (b) alveoli are exposed to NO. In one embodiment, less than 90% of poorly ventilated (a) areas of the lung or (b) alveoli are exposed to NO.

iNO delivered according to the present invention may be useful in treating various lung conditions, including PAH. Co-administration of iNO with pharmaceutical compositions useful for treating hypertension (such as a vasodilator) produce sustained effects of such pharmaceutical compositions in a patient, but do not cause a synergistic effect such that the patient becomes hypotensive.

Administration of Vasodilators including PDE-5 Inhibitors for Treatment of Pulmonary Arterial Hypertension

Vasodilators are typically administered for treatment of hypertension. Vasodilators include PDE-5 inhibitors and stimulators of soluble guanylate cyclase, such as riociguat. PDE-5 inhibitors such as sildenafil and tadalafil have been shown to improve clinical status, exercise capacity, and hemodynamics in PAH patients (Montani, D., et al., Adv. Ther., 2009 September:26(9):813-825). Sildenafil is approved to treat PAH (REVATIO®). However, REVATIO® is contraindicated for use with organic nitrates or riociguat (see Contraindications on label/package insert for REVATIO®) because of the risk of hypotension.

Some PAH patients being treated with a PDE-5 inhibitor such as REVATIO® will also need a secondary drug regimen to enhance the treatment of the disease. The inventors have demonstrated that inhaled nitric oxide (iNO) works locally in the lungs to enhance vasodilation without causing systemic hypotension in the patient when administered in conjunction with another vasodilator.

In an embodiment of the invention, co-administration of a pharmaceutical composition to treat hypertension (i.e., an anti-hypertensive agent) and iNO according to the present invention does not have any additive hemodynamic effect on a patient. In another embodiment, co-administration of iNO and an anti-hypertensive agent does not have a systemic additive hemodynamic effect on a patient. In yet another embodiment, iNO delivered to the lungs has a protective effect on the patient in preventing hypotension that may occur with administration of systemic vasodilators. In another embodiment of the invention, iNO administered to the lungs in accordance with the present invention while a patient is on a vasodilative treatment regimen for hypertension blunts the hypotension that may occur with administration of systemic vasodilators. In another embodiment, administration of iNO to the lungs while a patient is on a vasodilative treatment regimen for hypertension demonstrates no further significant decrease in systolic and/or diastolic blood pressure. In another embodiment, administration of iNO to the lungs while a patient is on a vasodilative treatment regimen for hypertension does not cause hypotension or symptoms of hypotension such as syncope or lightheadedness. In an embodiment of the present invention, iNO is rapidly absorbed by hemoglobin, resulting in low levels of circulating NO metabolites, which results in no additive hemodynamic effect on blood pressure.

In an embodiment of the present invention, administration of iNO to the lungs according to the present invention has only a local vasodilatory effect. Stated another way, administration of iNO to the lungs according to the present invention does not have a systemic effect on vasodilation. This is true in patients receiving both acute and chronic administration of iNO. This preferential local vasodilation in the lungs allows administration of iNO to be safe and effective even in combination with administration of an anti-hypertensive agent, such as a vasodilator.

In an embodiment of the invention, the anti-hypertensive agent is a vasodilator. In one embodiment, the anti-hypertensive agent is a PDE-5 inhibitor. In an embodiment of the invention, the PDE-5 inhibitor is sildenafil, tadalafil, vardenafil or salts thereof. A marketed example of each of these PDE-5 inhibitors is REVATIO® (sildenafil), CIALIS® (tadalafil), LEVITRA® (vardenafil) and STAXYN® (vardenafil). In an embodiment of the invention, the dosages for each of these vasodilators/anti-hypertensive agents is any one or more of the marketed dosages. In an embodiment of the invention, the dose for the PDE-5 inhibitor is from about 1 mg to about 200 mg, from about 2.5 mg to about 200 mg, from about 5 mg to about 200 mg, from about 10 mg to about 200 mg, from about 15 mg to about 200 mg, from about 20 mg to about 200 mg, from about 25 mg to about 200 mg, from about 50 mg to about 200 mg, from about 100 mg to about 200 mg, or from 150 mg to about 200 mg. In an embodiment of the invention, the dose for the PDE-5 inhibitor is 1 mg, 2.5 mg, 5 mg, 10 mg, 15 mg, 20 mg, 25 mg, 50 mg, 100 mg, or 200 mg.

In another embodiment of the invention, the PDE-5 inhibitor is a non-specific PDE-inhibitor. In another embodiment, the non-specific PDE-5 inhibitor is selected from the group consisting of theophylline and dipyridamole. In an embodiment of the invention, the anti-hypertensive agent is a stimulator of soluble guanylate cyclase. In another embodiment of the invention, the anti-hypertensive agent is riociguat. One example of riociguat is ADEMPAS®, which is marketed by Bayer. ADEMPAS® is available in several doses, include 0.5 mg, 1.0 mg, 1.5 mg, 2 mg, and 2.5 mg.

In an embodiment of the invention, iNO is administered according to the pulsed manner discussed herein. In an embodiment of the invention, the iNO is delivered to a patient using the INOpulse® device (Bellerophon Therapeutics). In one embodiment, the patient is administered iNO for a period of at least about 12 hours, 13 hours, 14, hours, 15 hours, 16 hours, 17 hours, 18 hours, 19 hours, 20 hours, 21 hours, 22 hours, 23 hours, or 24 hours per day for a period of at least about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks or 20 weeks. In one embodiment, the patient is administered iNO for 8 weeks. In another embodiment, the patient is administered iNO for 16 weeks. In an embodiment of the invention, a nitric oxide therapy session occurs over a timeframe. In one embodiment, the timeframe is at least about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours per day.

In an embodiment of the invention, a nitric oxide treatment is administered for a timeframe of a minimum course of treatment. In an embodiment of the invention, the minimum course of treatment is about 10 minutes, about 15 minutes, about 20 minutes, about 30 minutes, about 40 minutes, about 50 minutes, about 60 minutes, about 70 minutes, about 80 minutes, or about 90 minutes. In an embodiment of the invention, the minimum course of treatment is about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours. In an embodiment of the invention, the minimum course of treatment is about 1, about 2, about 3, about 4, about 5, about 6, or about 7 days, or about 1, about 2, about 3, about 4, about 5, about 6, about 7, or about 8 weeks, or about 1, about 2, about 3, about 4, about 5, about 6, about 7, about 8, about 9, about 10, about 11, about 12, about 18, or about 24 months.

In an embodiment of the invention, the iNO is administered at anywhere from 10 mcg/kg ideal body weight (IBW)/hr to 100 mcg/kg IBW/hr or more. In one embodiment, the iNO is administered at 25 mcg/kg IBW/hr. In one embodiment, the iNO is administered at 30 mcg/kg IBW/kg. In one embodiment, the iNO is administered at 35 mcg/kg IBW/kg. In one embodiment, the iNO is administered at 40 mcg/kg IBW/kg. In one embodiment, the iNO is administered at 45 mcg/kg IBW/kg. In one embodiment, the iNO is administered at 50 mcg/kg IBW/kg. In one embodiment, the iNO is administered at 55 mcg/kg IBW/kg. In one embodiment, the iNO is administered at 60 mcg/kg IBW/kg. In one embodiment, the iNO is administered at 65 mcg/kg IBW/kg. In one embodiment, the iNO is administered at 70 mcg/kg IBW/kg. In one embodiment, the iNO is administered at 75 mcg/kg IBW/kg. In one embodiment, the iNO is administered at 80 mcg/kg IBW/kg. In one embodiment, the iNO is administered at 85 mcg/kg IBW/kg. In one embodiment, the iNO is administered at 90 mcg/kg IBW/kg. In one embodiment, the iNO is administered at 95 mcg/kg IBW/kg. In one embodiment, the iNO is administered at 100 mcg/kg IBW/kg.

In an embodiment of the invention, the patient is also administered oxygen with the iNO and the vasodilator. In an embodiment of the invention, the oxygen is administered at up to 20L/minute. In an embodiment of the invention, the oxygen is administered at up to 1L/minute, 2L/minute, 3L/minute, 4L/minute, 5L/minute, 6L/minute, 7L minute, 8L/minute, 9L/minute, 10L/minute, 11L/minute, 12L/minute, 13L/minute, 14L/minute, 15L/minute, 16L/minute, 17L/minute, 18L/minute, 19L/minute, or 20L/minute. In an embodiment of the invention, oxygen is administered as prescribed by a physician.

While preferred embodiments of the invention are shown and described herein, such embodiments are provided by way of example only and are not intended to otherwise limit the scope of the invention. Various alternatives to the described embodiments of the invention may be employed in practicing the invention.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Drug-Drug Interaction Study Between Pulsed, Inhaled Nitric Oxide and Sildenafil in Healthy Volunteers

Inhaled Nitric Oxide (iNO) has been approved for use in infants with hypoxic respiratory failure. Nitric oxide is a selective pulmonary vasodilator, whose action is mediated by the cyclic guanosine monophosphate (cGMP) pathway. Hence, studies were conducted to assess the efficacy and safety of iNO for use in adults with Pulmonary Arterial Hypertension (PAH). Patients that have PAH may already be on 2-3 drugs to treat PAH, such as sildenafil. The objective of this study is to investigate the potential pharmacodynamic interaction between pulsed iNO and sildenafil in healthy volunteers.

Five healthy volunteers received sildenafil for 24 hours prior to the addition of iNO. Changes in pharmacodynamics parameters such as Heart Rate, Blood Pressure, and Oxygen Saturation after dosing with both drugs was assessed for 27 hours and hourly for 4 hours post iNO discontinuation.

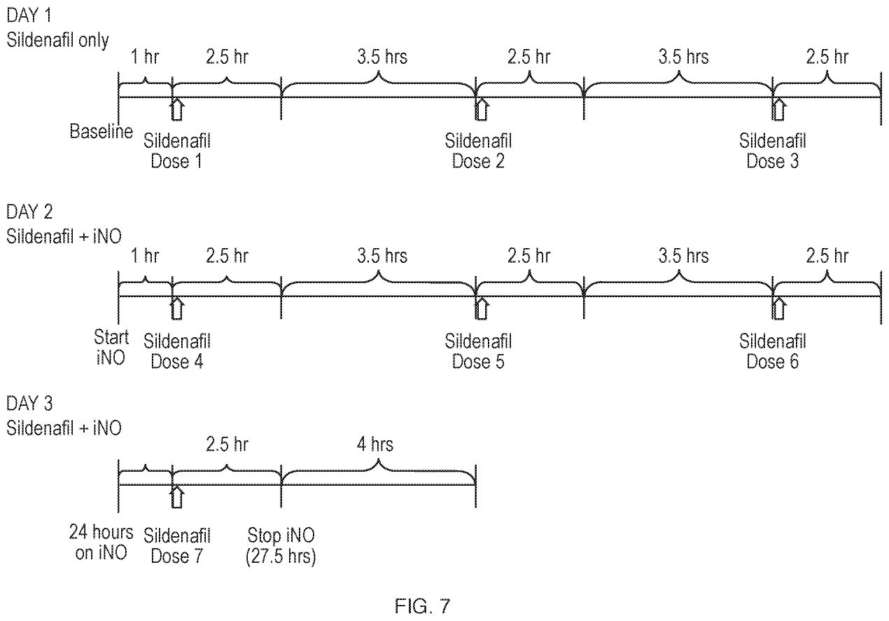

FIG. 7 illustrates the protocol design for this study. Briefly, on Day 1, subjects were dosed with sildenafil alone. One dose of 20 mg sildenafil was administered every 6 hours on day 1 for a total of 3 doses on day 1. Vital signs (heart rate, blood pressure, and oxygen saturation) were assessed beginning 1 hour prior to administration of sildenafil and every 30 minutes until 2.5 hours after each dose of sildenafil. Adverse events were also monitored. On days 2-3, the sildenafil regimen was continued as administered on day 1, and supplemented with 27.5 hours of pulsed iNO therapy at 75 mcg/kg individual body weight (IBW)/hr (INOPulse). Vital signs were assessed beginning 1 hour prior to administration of iNO and every 30 minutes until 2.5 hours after each dose of sildenafil. Upon discontinuation of iNO, vital signs were measured hourly for a 4-hour period, and subjects were monitored for syncope and lightheadedness. Nitric oxide metabolites were measured throughout administration of iNO and for 4 hours post-discontinuation of iNO. Drug and/or iNO therapy was discontinued if any subject's blood pressure dropped to less than 90/50 mmHg, and iNO therapy was discontinued if syncope or lightheadedness was observed.

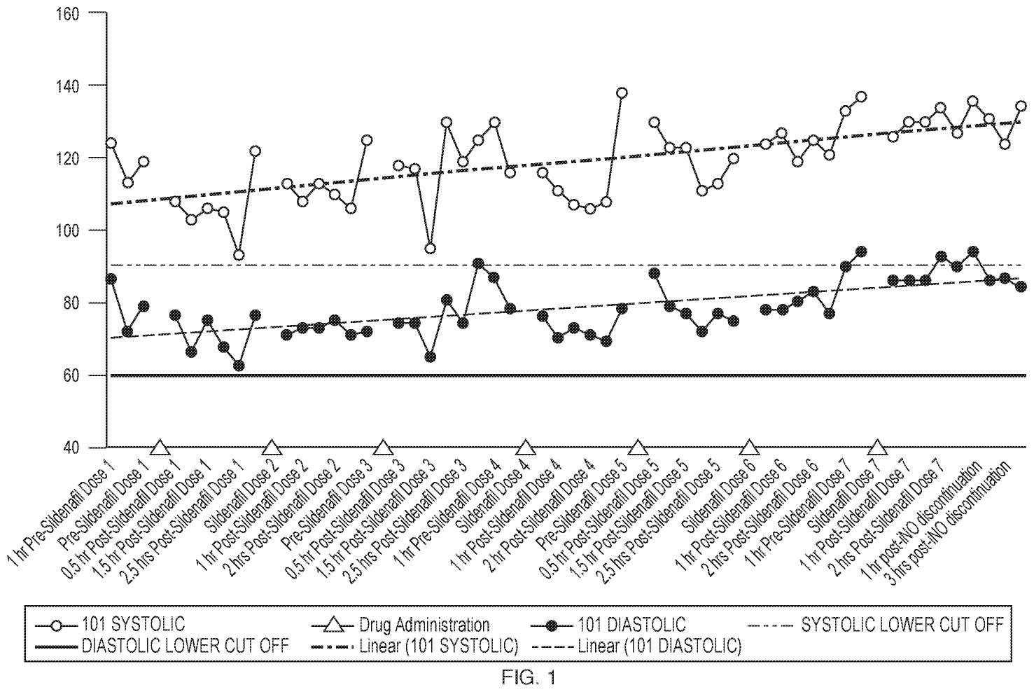

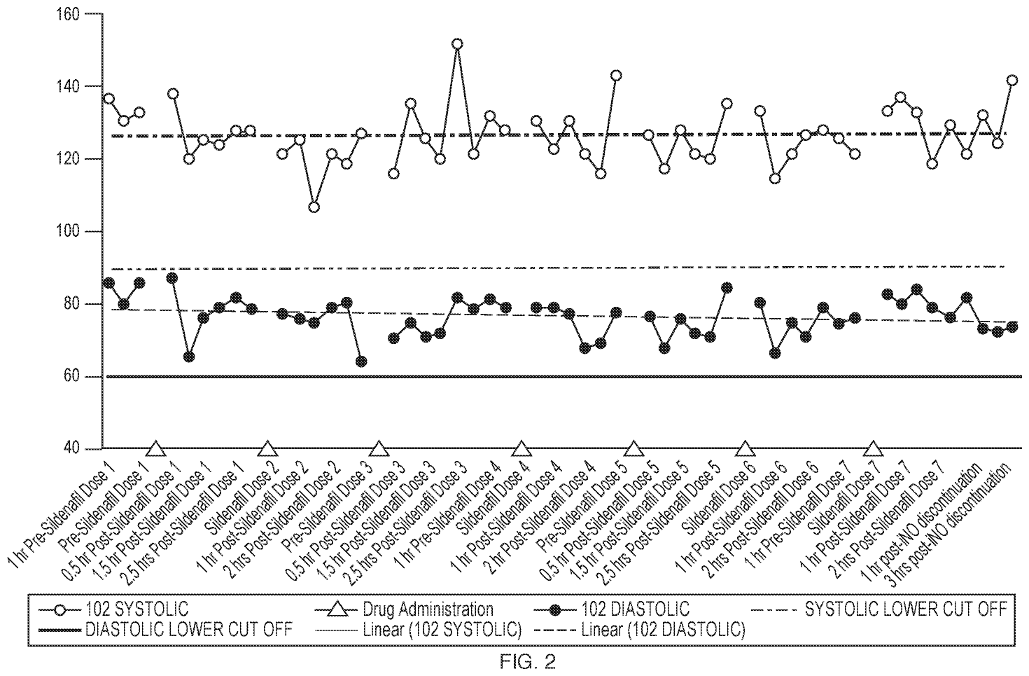

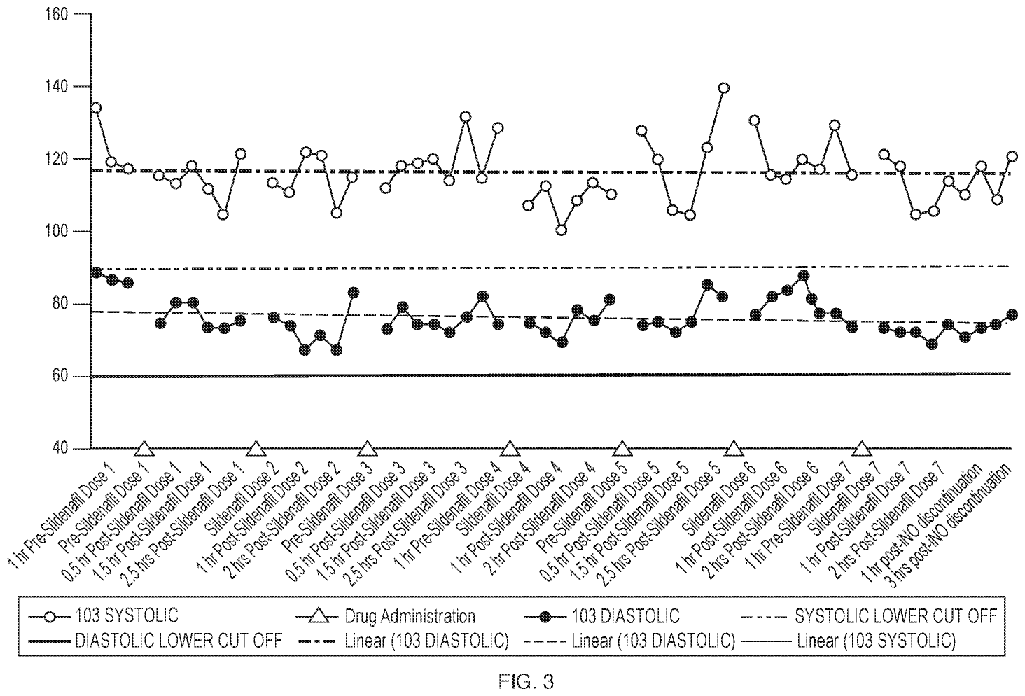

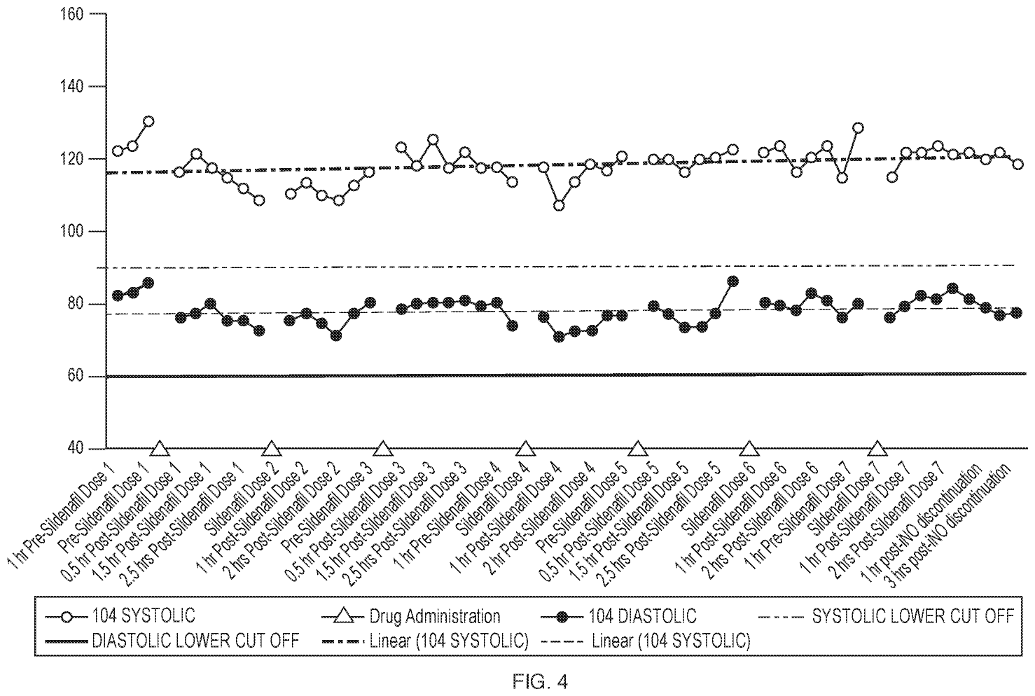

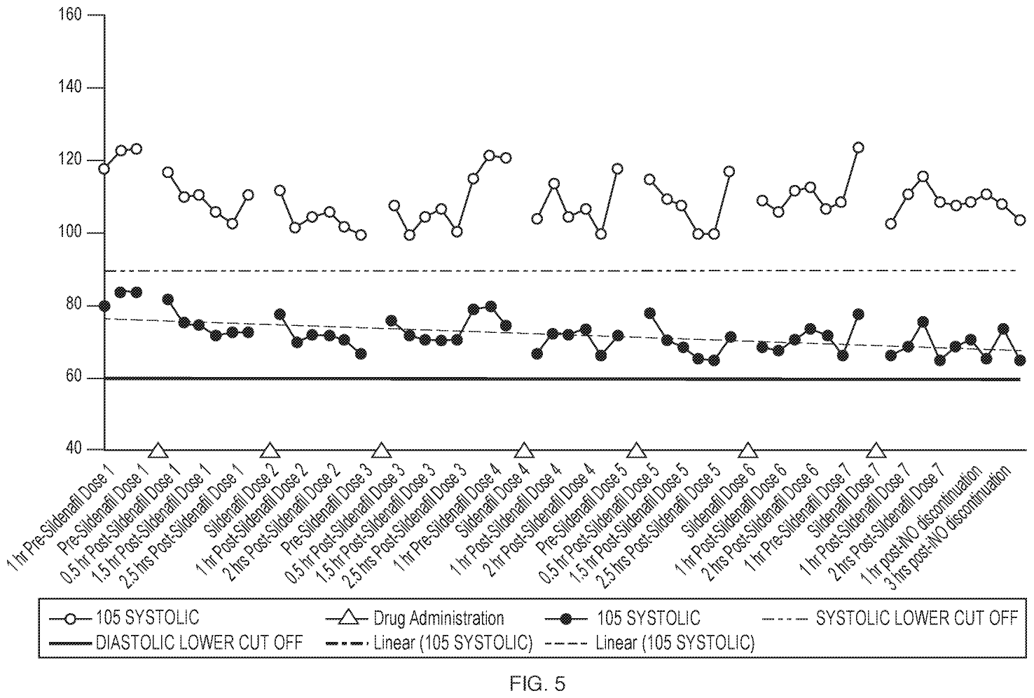

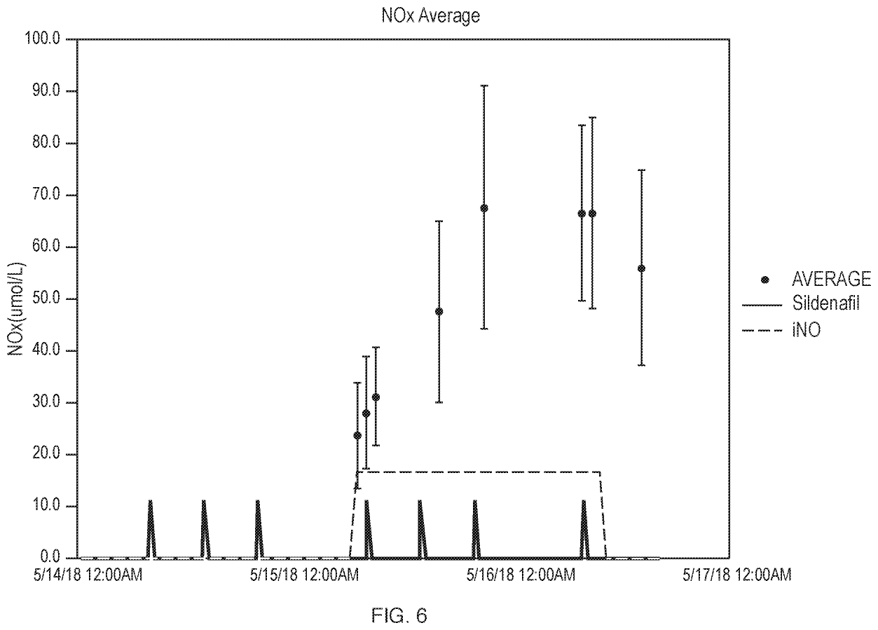

FIGS. 1-6 illustrate the results for this study. FIGS. 1-5 show the systolic and diastolic blood pressure results for each of subjects 1-5 at various intervals throughout the dosing period. A drop in blood pressure was measured in all 5 subjects within 2.5 hours after being dosed with sildenafil alone (all 3 doses on day 1). Similar drops in blood pressure were measured in all 5 subjects after each sildenafil dose on Days 2 and 3. The results demonstrate that there was no further decline in the systolic and diastolic blood pressures during co-administration of iNO (sildenafil doses 4 and 5) and at peak levels of nitric oxide metabolites (sildenafil doses 6 and 7), compared to the blood pressure values observed at the first 3 doses of sildenafil alone. Results were consistent across all subjects. No synergistic interaction (i.e., no hypotensive drug-drug interaction result) was observed between iNO and sildenafil, even at peak levels of metabolites in all volunteers in this study. There were no instances of syncope, lightheadedness, or signs of possible rebound upon discontinuation of iNO, and no other adverse events were identified.

### Example 2: Drug-Drug Interactions Between Pulsed, Inhaled Nitric Oxide and Riociguat in Healthy Volunteers

Ten healthy volunteers are recruited to investigate the potential pharmacodynamics interaction between riociguat and pulsed iNO. Subjects receive 2.5 mg riociguat three times per day for 5 days to achieve a steady state. Vital signs (heart rate, blood pressure, and oxygen saturation) are assessed beginning 1 hour prior to administration of the first dose of riociguat and every 30 minutes until 2.5 hours after each dose of riociguat. Adverse events are also monitored. On day 6, the riociguat regimen is continued as administered on previous days, except that pulsed iNO therapy begins 1 hour prior to the first dose of riociguat on day 6. Pulsed iNO therapy continues for 27.5 hours at 75 mcg/kg individual body weight (IBW)/hr (INOPulse). Vital signs are assessed beginning 1 hour prior to administration of iNO and every 30 minutes until 2.5 hours after each dose of riociguat. Upon discontinuation of iNO, vital signs were measured hourly for a 4-hour period, and subjects were monitored for syncope and lightheadedness. Nitric oxide metabolites were measured throughout administration of iNO and for 4 hours post-discontinuation of iNO. Drug and/or iNO therapy is discontinued if any subject's blood pressure dropped to less than 100/50 mmHg, and iNO therapy is discontinued if syncope or lightheadedness is observed.

We claim:

1. A method for preventing an additive hemodynamic effect when treating a patient with a therapeutically effective amount of a pharmaceutical composition for treatment of hypertension, the method comprising co-administering with said pharmaceutical composition a therapeutically effective amount of inhaled nitric oxide.

2. The method of claim 1, wherein the nitric oxide is delivered in a pulsatile manner by:
a. Detecting a breath pattern in said patient including a total inspiratory time;
b. Correlating the breath pattern with an algorithm to calculate the timing of administration of the dose of nitric oxide; and
c. Delivering the nitric oxide to said patient in a pulsatile manner over a portion of the total inspiratory time.

3. The method of claim 1, wherein the pharmaceutical composition is a PDE-5 inhibitor.

4. The method of claim 3, wherein the PDE-5 inhibitor is sildenafil.

5. The method of claim 3, wherein the PDE-5 inhibitor is tadalafil.

6. The method of claim 3, wherein the PDE-5 inhibitor is vardenafil.

7. The method of claim 3, wherein the PDE-5 inhibitor is a non-specific PDE-5 inhibitor.

8. The method of claim 7, wherein the non-specific PDE-5 inhibitor is selected from dipyridamole and theophylline.

9. The method of claim 1, wherein the pharmaceutical composition is riociguat.

10. The method of claim 1, wherein delivery of the dose of nitric oxide occurs within the first half of the total inspiratory time.

11. The method of claim 1, wherein the nitric oxide is delivered in a series of pulses over a period of time.

12. A method for preventing a systemic, additive decrease in blood pressure in a patient being treated for hypertension while improving the overall treatment of said hypertension, said method comprising co-administering a PDE-5 inhibitor and inhaled nitric oxide.

13. The method of claim 12, wherein the nitric oxide is delivered by:
a. Detecting a breath pattern in said patient, said breath pattern having a total inspiratory time and a total expiratory time;
b. Correlating the breath pattern with an algorithm to calculate the timing of administration of a dose of nitric oxide; and
c. Delivering said dose of nitric oxide to said patient in a pulsatile manner over a portion of the total inspiratory time for a period of time required for a therapeutically effective amount of nitric oxide to be delivered to said patient.

14. The method of claim 12, wherein the PDE-5 inhibitor is sildenafil.

15. The method of claim 12, wherein the PDE-5 inhibitor is tadalafil.

16. The method of claim 12, wherein the PDE-5 inhibitor is vardenafil.

17. The method of claim 12, wherein the PDE-5 inhibitor is a non-specific PDE-5 inhibitor.

18. The method of claim 17, wherein the non-specific PDE-5 inhibitor is selected from dipyridamole and theophylline.
